(19) United States
(12) Patent Application Publication
(10) Pub. No.: US 2025/0216388 A1
(43) Pub. Date: Jul. 3, 2025

(54) Determination of Components in Samples

(71) Applicant: IDEXX Laboratories, Inc., Westbrook, ME (US)

(72) Inventors: Jeremy Hammond, Standish, ME (US); James Russell, North Yarmouth, ME (US); Jason Aguiar, Freeport, ME (US); Kayla Carpenter, Gorham, ME (US); JuiMing Lin, Falmouth, ME (US)

(21) Appl. No.: 18/989,394

(22) Filed: Dec. 20, 2024

Related U.S. Application Data
(60) Provisional application No. 63/615,643, filed on Dec. 28, 2023.

(51) Int. Cl. G01N 33/569; G01N 33/53

(52) U.S. Cl. CPC G01N 33/56911; G01N 33/5308; G01N 2469/10

(57) ABSTRACT

An apparatus and method for detecting the type, presence or amount of a component in a sample. The apparatus and method include a multi-section container having multiple depths and imaging the depths at selected sets of sample Z-axis heights, X-Y coordinates, or both, within the sections after a predetermined time to allow for components in the sample to settle.

### CROSS-REFERENCE TO RELATED APPLICATIONS

This application claims priority to U.S. Provisional Application Ser. No. 63/615,643, filed Dec. 28, 2023, which is incorporated by reference in its entirety

### FIELD

The present disclosure relates generally to the determination of components in biological, industrial or environmental samples.

### BACKGROUND

Methods of determining components in various types of samples suffer from technical challenges. For example, manual microscopic analysis of ear cerumen, peripheral blood, and fine needle aspirates are often analyzed for sample components by smearing the samples on glass slides. Each sample type has varying viscosity, element concentration and complicating interferences (e.g., lipids, wax, clumped cells, tissue). These factors as well as technician skill lead to frequent slide preparation difficulties and inconsistencies that can hinder efficacy of interpreting these samples via manual microscopy.

Accordingly, there is a need for a simple and accurate method for quickly and accurately identifying components in samples.

### SUMMARY

One aspect of the present disclosure is directed to a method for identifying a component in a liquid sample. The method includes diluting a native sample or non-dissolving components thereof with a dilution liquid to provide a liquid sample having a uniform component distribution; introducing the liquid sample into a sample container comprising a first section comprising a first Z-axis height and second section comprising a second Z-axis height that allow for settling of components in the liquid sample; allowing the components in the liquid sample to settle for a predetermined amount of time; and imaging a Z-axis field of view (FOV) comprising an increment of the Z-axis height of the liquid sample at a first location within the first section and a second location within the second section, wherein the first location comprises a first set of X-axis and Y-axis coordinates and the second location comprise second set of X-axis and Y-axis coordinates.

In another aspect, the native sample may be a highly viscous or semi-solid sample biological, environmental or industrial sample. For example, the native sample comprises a fine needle aspirate of a tissue. The tissue may be cells from a lymph node, a cutaneous or subcutaneous mass or an organ. The native sample may also be ear cerumen, a fecal sample, a lung lavage, a fluid from an effusion, a blood sample, synovial fluid, thoracic fluid, abdominal fluid, cerebrospinal fluid or a urine sample.

In another aspect of the method of the disclosure, the settling of non-bacterial components in the liquid sample is complete after the predetermined amount of time, or alternatively, the settling of non-bacterial components in the liquid sample is incomplete after the predetermined amount of time.

In further aspects of the disclosure, the Z-axis heights of the first section and the second section may be independently between about 0.1 to 0.5 mm, and/or a depth of field of the increment of the Z-axis height may be about 1 μm to 10 μm, and/or a Z-axis height of the first section may be at least 50 μm more than the Z-axis height of the second section.

In various aspects of the disclosure, imaging may be conducted with a confocal microscope or and epifluorescence (EPI) microscope, the increment of the Z-axis height may include a bottom of the first section or the second section of the container, the imaging may include imaging FOVs at a plurality of Z-axis increments in each section, and/or the imaging may include imaging FOVs at a plurality of X-Y coordinates in each section.

In additional aspects, the number of Z-axis FOVs is determined by at least one of the component being analyzed, the dilution ratio and a Z-axis height, and optionally, the number of Z-axis FOVs is further determined by a predetermined image acquisition time. In addition, the statistically analyzing may include correlating a type, presence or amount of a component in the plurality of FOVs to the type, presence or amount of the component in the liquid sample. The statistically analyzing may include correlating a type, presence or amount of a component within a FOV by comparing an image from the FOV to a predetermined image indicative of the type, presence or amount of the component.

In a next aspect, the imaging may include determining a fluorescent response of a stained component in the FOV, and may include obtaining a bright-field image of the FOV.

### BRIEF DESCRIPTION OF FIGURES

The accompanying drawings, which are included to provide a further understanding of the disclosure, are incorporated in and constitute a part of this specification, illustrate embodiments of the disclosure, and together with the detailed description serve to explain the principles of the disclosure. No attempt is made to show structural details of the disclosure in more detail than may be necessary for a fundamental understanding of the disclosure and various ways in which it may be practiced.

FIG. 1 shows a schematic representation of an apparatus according to an aspect of the disclosure.

FIG. 2 is a diagram of example components of an apparatus or system, in accordance with aspects of the present disclosure;

FIG. 3 is a diagram of an example of data relating to number of fields of view, in accordance with aspects of the present disclosure;

FIG. 4 is a flow diagram of an example of an operation for capturing images, in accordance with aspects of the present disclosure;

FIG. 5 is a diagram of an example of data relating to total time to count white blood cells, in accordance with aspects of the present disclosure;

FIG. 6 is a diagram of an example of components of a point-of-care apparatus, in accordance with aspects of the present disclosure.

FIG. 7 shows a confocal 3-D rendering image for fluorescence-stained cocci obtained according to a method of the disclosure.

FIG. 8 shows a confocal 3-D rendering image for fluorescence-stained bacterial rods according to a method of the disclosure.

FIG. 9 shows 3-D rendering images for bacteria and dust dispersion in a liquid sample after a settling time of 20 minutes.

FIG. 10 shows bright field and fluorescent images of a blood sample obtained according to a method of the disclosure.

### DESCRIPTION

In its various aspects, the disclosure is directed to apparatuses, methods, reagents, containers and kits for analyzing a variety of sample types from animals, industry, and the environment. The aspects of the disclosure use combinations of diluents, buffers, and staining agents to homogenously suspend the non-dissolving sample components (e.g., cells, debris, mites) of each sample type in a liquid sample confined to application-specific sample container that can be imaged with a digital microscopy system. In various aspects, sample containers may have distinct sections with different heights that allow for settling of selected sample components in the liquid after different amounts of times.

The various aspects of the disclosure allows for preparation standardization across multiple sample types and ensure analysis of a homogenous distribution of sample components in a liquid sample that is analyzed according to the disclosure. The various aspects of the disclosure allow for the maintenance of sample components in their native state by avoiding mechanical stresses typically associated with processing highly viscous or semi-solid samples (e.g., smearing, staining, sphering) and the flow or other constraints of a sample cartridge/system. Therefore, extensive numbers/volumes of sample components can be analyzed with greater statistical accuracy. Components from a native sample that are uniformly suspended in diluents (e.g. containing buffers and stains) will settle through the measurement chamber such that statistical sampling throughout any chosen volume will yield consistent performance. Analysis of the settling or settled components can be conducted by imaging the chamber bottom for all settled elements and/or within the depth of fluid for slower settling, floating or unsettled elements.

Sample types may have specific dilution ratios and liquid sample container volumes to ensure there is no cellular overlap, interference and/or crowding where critical count and distribution statistics are applied. Multiple liquid sample container depths will have the effect of diluting the sample components in shallower depth portions of a sample container or concentrating the components with deeper depth portions of sample container resulting in linear change in component concentration throughout the various container depths, which can be used to account for low or high concentration samples. For example, the concentration of cells and/or dilution factor of the biological sample can be matched with an appropriate height of the reservoir to optimize settling time (e.g., 1 micron per second for blood cells). The settling time of a biological sample can be longer or shorter depending on the specific gravity of the diluent and size and/or density of the cells. In example implementations, if one or more components of the biological sample (e.g., cells of interest) have a low concentration, a higher reservoir height can be more beneficial as a user can wait for those one or more components (e.g., cells) to settle. Once the one or more components (e.g., cells) have settled, the settled one or more components (e.g., cells) may have a higher concentration at one or more locations within the reservoir, for example, on or near the bottom of the reservoir. When imaging the biological sample, this arrangement and protocol can improve imaging and/or other diagnostics by, for example, requiring fewer fields of view in to achieve a desired number of one or more components (e.g., cells) present in an image. A calculation is based on a standard concentration of cells per unit volume (microliter for example) multiplied by the actual volume in the container to get the count that will be on the bottom. In some embodiments of the disclosure, statistical sampling for expected component concentrations may use a fixed, predetermined, number of field of views (FOVs) within the sample volume based upon selected sampling statistics to ensure limit of detection as well as accuracy.

As used herein, the term “sample” may refer to either an unprocessed (“raw” or “native”) sample in any form (fresh, frozen, etc), or “sample” may refer to a native sample that has been processed for analysis according to the disclosure (e.g., a processed sample), the difference between the samples apparent from the context of the disclosure.

As used herein, the phrases “for example”, “as an example”, and the like do not mean “preferred” and simply refer to an example aspect of the disclosure unless the context clearly indicates otherwise. Although the disclosure is not limited in this regard, the terms “plurality” and “a plurality” as used herein may include, for example, “multiple” or “two or more.” The terms “plurality” or “a plurality” may be used throughout the specification to describe two or more components, devices, elements, units, parameters, or the like.

As used herein, the term “imaging device” refers to and means any device that is configured to sense at least the visible light spectrum and to provide an image. An imaging device may include components such as, without limitation, one or more lenses and a sensor.

As used herein, the term “field of view” refers to and means a region that is capturable by an imaging device. The term “working distance” refers to and means the object to lens distance where the image is at its sharpest focus. An image can be said to be focused on a scene at the working distance. The term “depth of field” refers to and means the distance between the nearest and furthest elements in a captured image that appear to be acceptably in focus. Depth of field and what is considered “acceptable” focus will be understood in the field of optical imaging. The term “resolving power” refers to the smallest distance between two features that an imaging device can clearly present as being separate features.

As used herein, the term “dilution ratio” refers to and means a ratio of volume of diluent to volume of biological sample. Accordingly, a ratio of volume of diluent to volume of biological sample of 75:1 may be described as a dilution ratio of 75:1. A diluent may be and include any substance or combination of substances that can be combined with a biological sample, including, without limitation, reagents, stains, buffers, and/or working fluids, among other possible substances.

The system detects and, optionally, quantifies components in collected samples when the sample components are suspended in a liquid diluent. Samples can include any type of biological, industrial and environmental samples to the extent that the sample components can be suspended in a liquid medium. Typical biological samples include blood, urine, saliva, cerebral-spinal fluid, tumor samples, and the like. In addition, solid, semi-solid, or highly viscous samples, as ear cerumen, peripheral blood and fine-needle aspirates (FNAs) can be suspended in a liquid medium and analyzed for components according to the methods of the disclosure. Other biological samples may include one or more of the following: fecal matter; secretion, excretion, lavage fluid; body cavity fluids; semen; skin cells; biopsied samples, exotics; cultured cells, synovial fluid, abdominal fluid, cerebrospinal fluid, or urine.

As used herein, “non-dissolving sample components,” or simply “sample components” or “components,” include sample various types of sample contents that have sufficient mass to cause the contents to settle over a predetermined amount of time. Example sample components include, for example, cells (e.g., bacteria, blood cells, epithilial cells, tissue cells, cultured cells, mesenchymal cells), organisms (e.g., mites, fungus, yeast, parasites), and debris (e.g., dust, pollen, micparticles, crystals, casts). The microscopic analysis techniques of the disclosure can identify, quantify, and differentiate the various sample components in a processed sample. Dissolved sample substituents that do not settle are not sample components.

Aspects of the present disclosure relate to automated evaluation of a sample based on capturing images of the sample by an imaging device and analyzing the captured images. In aspects, the present disclosure evaluates components of the sample in order to reduce a clinician's workflow, reagent management, and run costs. Aspects of the present disclosure relate to completing the evaluation in a reasonable amount of time and with a reasonable degree of statistical confidence. As described below, various aspects of the present disclosure are implemented by combining a sample with a diluent and evaluating the combination in a sample chamber. The evaluation may capture a different number of images depending on the sample chamber depth dimension and/or the ratio of volume of diluent to volume of sample.

### Sample Processing and Staining

Biological samples from humans, animals, industry and the environment can be collected using known techniques. Solid, semi-solid, or highly viscous samples, such as ear cerumen, peripheral blood, and fine needle aspirates, provide unique challenges when preparing the sample to be analyzed on, for example, glass slides. For instance, each has varying viscosity, component concentration and complicating interferences (e.g., lipids, wax, clumped cells, tissue). These factors as well as technician skill lead to frequent slide preparation difficulties and inconsistencies that can hinder efficacy of interpreting these samples via manual microscopy. The most common problems with manual slide preparation are the lack of a true monolayer, mechanical damage to cells, lack of homogeneity, and poor or inconsistent staining. Additionally, in typical manual glass slide microscopy analysis, small component assessment can be challenging amongst larger components that are in the same coincident space. Ear cerumen (ear wax) in particular is a difficult sample because it is inconsistent (very hard to very soft), contains multiple constituents and contaminants, and is typically available in a small amount.

In order to address these and other sample analysis challenges, the present disclosure includes the use of stabilizing diluents, buffers and surfactants for preserving native cell morphology and assuring homogenous distribution of non-dissolving sample components within a processed sample that is analyzed according to the disclosure. A native sample, once processed as described herein to be in liquid form, can be added to a specified volumetric microscopy container that provides an alternative to typical manual microscopy preparation challenges and leads to preparation standardization and superior accuracy.

As one example, native samples can be obtained, for example, from bacteria culture (e.g., agar plates and/or liquid media), bacteria infected fluid, and tissue swabs or collection devices. The native samples can be suspended in an isotonic buffer solution (suspension liquid) with neutral pH to provide a processed sample containing a homogeneous mixture of non-dissolvable sample components. Embodiments to facilitate the suspension of the non-dissolving sample components into a homogeneous liquid sample include vortexing the sample for a few (e.g., 10) seconds to break apart aggregated components or extract components from the sample collection devices with our without the addition of surfactant (e.g., 0.01% of Tween 20™ (polysorbate 20)), and incubating the liquid samples in a 37° C. water bath for a short period of time (e.g., one minute).

Surfactants in a sample suspension liquid can facilitate the suspension process, compromise cell wall, and facilitate the stain uptake. Example surfactants can include cationic, anionic, non-ionic, and zwitterionic types of chemicals. Cationic surfactant are substances that bear positive charges such as docosyltrimethylammonium chloride; anionic surfactant are substances that bear negative charges such as Sodium dodecyl sulfate (SDS); nonionic surfactants contain no charge such as Tween 20™ (polysorbate 20); zwitterionic surfactants belong to the class of surfactants that is composed of both positive and negative charges such as Cocamidopropyl betaine. Surfactant concentration in a liquid sample can range from about 0.001% to about 0.5%. For example about 0.001%, 0.005%, 0.01%, 0.05%, 0.1%, 0.2%, 0.3%, 0.4% and 0.5%.

A suspension liquid may include pH buffering agents and salts (sodium chloride, Tris-HCl (Tris(hydroxymethyl)aminomethane hydrochloride)), Tris-base (Tris(hydroxymethyl)aminomethane)), and EDTA (Ethylenediaminetetraacetic acid)). Examples of the suspension liquid may include these buffering agents, but not limited to, vary from 10 mM to 200 mM for each buffering component. An example suspension liquid for processing native samples can include sodium chloride, Tris-HCL, EDTA, and Tris-base. In embodiments, example concentrations of these ingredients may include the following: about 6-10 g/L of sodium chloride, 1.4-2.0 g/L of Tris-HC1, 0.6-1.0 g/L of EDTA, and 2.0 to 1.76 g/L of Tris-base. An example combination of these ingredients includes the following: about 7.9 g/L of sodium chloride, 1.65 g/L of Tris-HC1, 0.8 g/L of EDTA, and 2.35 g/L of Tris-base.

Certain components in a sample can be fluorescently-labeled by methods known in the art, e.g. bacteria and white blood cells. For example, nucleic acid staining dyes and cell surface markers tagging by antibodies (e.g., anti-E. coli antibody, anti-Pseudomonas aeruginosa antibody, and the like) or aptamers (e.g., single-stranded DNA or RNA) conjugated with fluorescence probes. Bacteria cell wall permeable fluorescent nucleic acid staining dyes can be used, for example Acridine Orange or SYTO-13™ (both Thermo Fischer), or thiazole orange, along with other intracellular nucleic acid staining dyes.

In one aspect, nucleic acid staining efficiency includes staining variance associated with structural differences of cell walls when uptaking the staining dyes. Some universal nucleic acid stains do not achieve an optimal staining due to low fluorescent dye uptake. Therefore, to increase the staining efficiency, the samples can be incubated at elevated temperatures (e.g., water bath or an oven at 25-37° C., or a dedicated heating system). Alternatively or additionally, a low concentration of one or more surfactants can be added to the samples. The surfactants compromise the cell wall, which allows fluorescent dyes to diffuse rapidly while maintaining the shape of the cell wall. Both thermal and surfactant treatments can be combined to achieve an optimal staining condition.

Nucleic acid stains can provide a sufficient signal-to-noise ratio (SNR) to evaluate components (e.g. bacteria and WBC) in an assay designed to count such components. As one example, SYTO-13™ stain is a universal fluorescent stain for bacteria that can provide a signal to noise ratio sufficient for bacteria identification. Noise includes background fluorescence (autofluorescence) and any non-bacteria elements that may be fluorescing. Signal includes the magnitude of fluorescent intensity from bacteria. Sufficient SNR can occur when the bacteria signal is clearly separated from the noise signal. An example of a sufficient SNR is 2:1 of signal to noise, and higher values can enhance performance. In one example, presence and absence of components (e.g. bacteria and WBC) in the sample is determined when a threshold of fluorescent signal in the liquid sample solution is above the background noise floor. In an example involving bacteria, an E. coli. bacterial sample is introduced to a resuspension solution containing a cell permeable nucleic acid dye (Acridine Orange), 0.01% Tween 20™ (polysorbate 20), and 100 mM phosphate pH 7.5. The suspension solution can be incubated in a 37 C water bath to increase dye uptake.

### Sample Container

As described further herein, an epifluorescence (EPI) or confocal microscope can be set up to optically scan a sample container, such as a custom container, vessel, chamber, or slide (e.g., cell counter slide) made up with glass or plastics and, in some embodiments, without auto fluorescence. In embodiment, the container material is translucent or transparent.

Once the suspension liquid is mixed with the sample to provide a liquid sample, the mixture may be loaded into a sample container. In some aspects, microbubbles in the container are avoided and the container is completely filled. No seal over the loading port or vent is necessary if a sample is analyzed in a short amount of time (e.g., minutes) such that sample evaporation does not affect the analysis. A mineral oil may be used to seal a sample loading port and vent holes in the container to prevent sample evaporation (since the objects may move in any X-Y-Z motion during the image acquisition when water evaporation occurs) depending on the amount of time needed for analysis of container contents. Parafilm and other materials that effectively inhibit evaporation without interacting with or contaminating the sample may be used. Container/sample scanning run times of less than ten minutes are generally not affected by evaporation, but the mineral oil or similar seal may be considered for longer runs. Unless otherwise apparent from the context of the disclosure, the volume of the sample container and the sample are congruent such that the container is essentially completely full.

In various aspects of the disclosure the sample container will have multiple sections having sufficient depth that will allow the liquid sample components to separate and ultimately settle at the bases of the different sections of the container at different times. The depth of each section is referred to herein as section's Z-axis depth. When samples are prepared to provide for a homogeneous dispersion of sample components in a liquid sample, the components will settle at the bottom of each section of the container at different times depending on the Z-axis depth of the section. For instance, in “deeper” sections, i.e., those with larger Z-axis depth than “shallow” sections with smaller Z-axis depths, uniformly distributed sample components will take longer to settle on the bottom of the sections. In addition, each of the various sample components may have different settling rates depending on several factors including the viscosity of the sample, the charge of the components, the size of the components, and the density of the component with respect to the specific gravity of the sample fluid. For example, because bacteria are smaller, less dense, and have electric charge than most sample components, they will remain suspended in the liquid sample longer than most other components. Non-bacterial components usually settle out of solution within a few minutes or less such that the analysis of the liquid sample can be conducted in less than 10 minutes.

As further described herein, analysis of the sample includes scanning the sample components in the various sections of the container at different times and at different locations within the sample volume in each section, including the bottom of a section.

In embodiments of the disclosure, the sample container has two, three or more sections having Z-axis depths that are different from each other by at least 100 μm, at least 200 μm, at least 300 μm, at least 400 μm or at least 500 μm. The depth of the sections of the container of about 50 to about 1000 micrometers (micron or μm) provide sufficient depth that allow for settling of non-bacterial components within a time period desired for analysis in a clinical laboratory. For example, container section heights of 50 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm or 1000 μm may be used. The difference in height between container sections should be sufficient to allow for meaningful difference in the time it takes for components of a sample to settle to the bottom of a section. In some embodiments, the difference in section height is at least 50 μm, 100 μm, 150 μm or 200 μm so that sample components settle in a shallower section at a discernibly different time than components in a deeper section. For example, example predetermined times include 10 seconds, 20 seconds, 30 seconds, 40 seconds, 50 seconds, 60 seconds, 70 seconds, 90 seconds, 90 seconds, 100 seconds, 110 seconds, 120 seconds, 150 seconds, 180 seconds, 210 seconds, 240 seconds, 270 seconds, 300 seconds, 330 seconds, or 360 seconds.

In various aspects, the total volume of the sample within a container ranges from 5 μl to about 1000 μl, for example about 5 μl, 50 μl, 100 u1, 200 μl, 300 μl, 400 μl, 500 μl, 600 μl, 700 μl, 800 μl, 900 μl, or 1000 μl. As noted herein, the container may have multiple sections that in combination contain the entire sample volume.

In addition, some sample containers may have completely independent chambers, each with two or more Z-axis depths that can be used to analyze samples collected from different areas on an animal or other sources. For example, samples of cerumen from both ears from an animal can be separately analyzed in a container having independent chambers such that left and right ear samples can be independently analyzed.

An example commercially available container includes an InCyto C-Chip disposable slide (INCYTO), a SediVue Dx container (IDEXX Laboratories), or a Sight OLO container (Sight Diagnostics), which include an enclosed volume that can be filled and then analyzed with a microscope as disclosed herein. Other configurations can address system constraints and methods of heating as described herein. As another example, a “container” as described herein may include one or more of the components of the cartridge disclosed in U.S. Provisional Application No. 63/615,371, filed Dec. 28, 2023 (which is incorporated herein by reference in its entirety). For example, a “container” as described herein may be implemented as one or more “reservoirs” of the cartridges described in U.S. Pro. App. No. 63/615,371.

### Apparatus

In various aspects of the disclosure, epifluorescence (EPI) or confocal microscopy can be used to identify sample components. In addition, bright field microscopy can be used to identify the type of sample component. In embodiments of the disclosure, the apparatus includes bright-field light sources having different wavelengths and light sources that excite fluorescent dyes at different wavelengths. The number of fluorescent light sources and bright field light sources can vary depending on the expected contents of a sample to be analyzed.

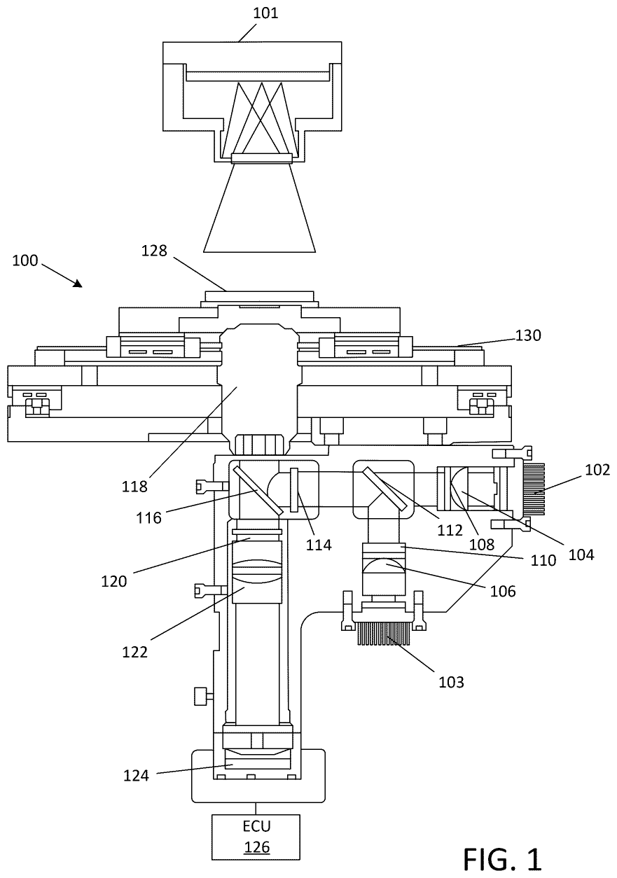

An example microscope apparatus of the disclosure is shown in FIG. 1. The apparatus 100 includes a digital microscope that includes a bright field light source 101, a fluorescent blue light source 102, a fluorescent ultraviolet light source 103, collector lenses 104 and 106, a blue excitation filter 108, an ultraviolet excitation filter 110, an excitation dichroic 112, a field lens 114, an imaging dichroic 116, an objective lens 118, a triband filter 120, a tube lens 122, a camera 124, and an electronic control unit (ECU) 126. The objective lens 118 may facilitate a bright field light source (not shown). A sample container 128 is shown positioned on X-Y stage 130.

In operation, the sample container 128 is placed along the optical path of the microscope and illuminated with the bright field light source 101, the fluorescent blue light source 102 and/or the fluorescent ultraviolet light source 103. Sequentially or simultaneously, one of the fluorescent blue light source 102 or the fluorescent ultraviolet light source 103 illuminates the sample through dichroic 116, and the bright field light source can be applied in conjunction with or separately from the fluorescent light sources The sample interacts with the incident light and then generates a fluorescent signal that moves back down through the dichroic 116 to the camera 124. Alternatively or in addition, the bright field source is transmitted through the sample and objective lens 118 to the optics to the camera 124. The image(s) captured by the camera 124 are transmitted to the ECU 126 for automated analysis, as disclosed herein.

For scanning the sample, a Z-axis field of view (FOV) or multi-FOVs can be selected based on the known or expected concentration of the components in the sample to be evaluated. Scanning of the container can include scanning the depth of the container (or an increment or increments thereof) at a single X-Y coordinate within the container/sample, scanning a single Z-axis depth at multiple X-Y coordinates of the container/sample, or scanning multiple Z-axis depths at multiple X-Y coordinates within the container/sample. Scanning can occur at one or more sections of the container having different Z-axis heights.

A Z-stack scan can cover a particular X-Y coordinate in a sample from the container bottom to the top in order to access the entire bulk fluid. Alternatively, a Z-stack scan can cover one or more increments of the Z-stack. Each increment of Z-axis depth may be set 1 to 50 μm based on the optics depth of field. For example, each increment may be 1 μm, 2 μm, 3 μm, 4 μm, 5 μm, or 6 μm, 10 μm, 20 μm, 30 μm, 40 μm or 50 μm, or within the range of 1 to 6μ, 2 to 5 μm, or 3-4 μm. Image acquisition can be either the X-Y motion first to cover all FOVs at a particular Z-axis depth, then follow Z-stack scanning or vice versa.

In various embodiments of the disclosure, the Z-stack scan covers between about 10% to about 100% of the Z-axis depth of the sample. For example, the Z-stack scan can cover 10%, 20%, 30%, 40%, 50%, 60%, 70%, 80%, 90% or 100% of the Z-axis depth of the sample. When the Z-stack scan covers less than 100% of the Z-axis depth of the sample, the scan can include contiguous or non-contiguous increments of the Z-stack. The scan may include the bottom of the sample, the top of the sample, neither, or both. In one embodiment, the scan includes contiguous increments accounting for about 25% to 75%, for example about 25%, 30%, 35%, 40%, 45% or 50%, 55%, 60%, 65%, 70% and 75% of the Z-axis depth without including the top or bottom of the sample. Z-axis depth can be selected such that it is at least above the point where the focus depth of field has immediate noise from settled white blood cells or other fluorescing cells, for example about 25-50 μm above the bottom of the sample container, for example 25 μm, 30 μm, 35 μm, 35 μm, 40 μm, 45 μm or 50 μm, above the bottom of the sample container. In samples with settled cells or other settled sample components that generate a large fluorescent signal, the Z-axis scan can be even further from the bottom of the container, or up to about 75 μm, e.g., 55 μm, 60 μm, 65 μm, 70 μm, or 75 μm, from the bottom of the container in order to avoid noise from settled components.

In various embodiments, because sample components may settle, less than the entire volume of the sample is scanned. As discussed above, sample components settle at different rates, with smaller components, such as bacteria remaining in suspension the longest. And some sample components may float. Therefore, after a predetermined period of time the sample, or a section or sections thereof can be scanned to avoid the top and bottom of the sample to avoid scanning increments containing sample components that have settled. The increments may be at least 25 μm, 50 μm, 75 μm, 100um or more off the bottom of the sample container or section thereof to avoid interference from sample components at or near the bottom of the sample container. In addition, some sample components may float (e.g., lipids, adipocytes, etc.). Therefore, scanning the top of the sample can be avoided.

As an example embodiment, images obtained 20-50 μm from the bottom of the container may be high enough depending on the depth of focus of the camera system. Similarly, the FOV can jump, for example, to 50, 75, or 100 microns depth and use those fixed depth locations for the analysis. Data generated from the full-scan experiments can be used to choose appropriate depths and settling times and build a correlation function from the abbreviated data set to determine the presence or amount of bacteria in a sample.

In embodiments containing multiple component types, both the bottom of the container and the sample contents can be imaged to ensure that settled components and components remaining in suspension are analyzed.

Within the X-Y plane, one or more X-Y coordinates within the sample can be scanned at one or more Z-depths. The size of a coordinate location area that can be scanned at particular increment of the Z-stack is determined by the size of the focus of the microscope. For example, X-Y area in a field of view may be in the range 1 to 30 megapixels, which reflects an area of about 0.1 to about 3.0 mm². In an example sample container having an X-Y plane of about 0.5 to about 1.0 cm², the number of focus spots that can be imaged in the plane with no or minimal overlap would be about 275-550. For homogenous samples in multi-depth containers, the component distribution at a particular Z-axis depth should be the same regardless of what section of the sample container is scanned unless sufficient time elapsed such that sample components have settled in a shallow section of a container yet still suspended in a deeper section.

Because sample components can be expected to be uniformly distributed with the scanned sample, only a portion of the sample must be scanned in order to determine the presence and or amount of various components in the sample. In some embodiments, the total volume of the sample that may be scanned may be between about 1% to about 25%, for example about 10%. For quantitation of components in the sample when less than 100% of the sample is scanned, the amount of a component in the portions of the sample that were scanned can be correlated to the amount of component in the entire sample. In some embodiments 1 to about 100 FOVs are analyzed, for example, 1, 2, 3, 4, 5, 6, 7, 8, 9, 10, 20, 30, 40, 50, 60, 70, 80, 90 of 100 FOVs. In some embodiments, the number of FOVs that are analyzed are less than 50, less than 40, less than 30, less than 20, less than 10, or less than 5 FOVs. In various aspects, the amount of sample required to be scanned depends on the volume of fluid interrogated and the limit of detection. For instance, depth of field of the camera and the X-Y dimension of an FOV provides the volume of one image, which can be used to determine the volume of sample or number of FOVs needed for statistical sampling.

The camera exposure time can vary in view of excitation light intensity, camera dynamic range and elapsed assay time. In some embodiments, camera exposure time is 50 ms, 100 ms, or 150 ms. Because the dye will intercalate into the bacteria or other sample components over time, fluorescence emission from the stained bacteria will increase over time. Therefore, camera exposure time may be shorter near the end of a scan compared to the beginning. In some embodiments, the camera exposure time decreases over the course of scanning a sample at several X-Y and/or Z-axis depths by at least 25%, 50%, or 75%. Also, increase sample temperature can impact the stain uptake. Increasing exposure time will increase the signal when it is low, and decreasing exposure time will help pull the signal down if it is saturating the sensor.

At a given stain concentration and temperature, along with the susceptibility of the bacteria to allow the stain to uptake, then the fluorescent signal will increase with time as the stain moves towards equilibrium (and potentially starts to decrease if the light source continues after the stain is used and the stain begins to photo-bleach). Imaging the sample after a predetermined amount of time allows for component settling and stain uptake. Therefore one aspect of the disclosure optimizes time to result even when stain and fluorescent concentration has not reached equilibrium before sample scanning begins. Accordingly, in one embodiment, the system may use multiple exposure times that allow the fluorescent response to increase over the scan time while avoiding saturated signals. In other words, the system may adjust exposure times to be longer when fluorescent signal is low (at the beginning of predetermined amount of time, and shorter when fluorescent signal is higher (near the end of the predetermined amount of time). In various embodiments of the disclosure the exposure time can range from about 0.5 to about 100 ms, 0.5 ms to about 20 ms, 50 milliseconds (ms) to about 100 ms, or about 20 ms to about 140 ms. Example exposure times during a scan of a sample may be one or more of 0.5 ms, 1 ms, 5, ms, 10 ms, 15 ms, 20 ms, 30 ms, 40 ms, 50 ms, 60 ms, 70 ms, 80 ms, 90 ms, 100 ms, 110 ms and 120 ms, which may take into account camera response time, aperture size, and variations in fluorescent signal over time as noted above.

Changing X-Y coordinates of the Z-stack FOV in the sample can be accomplished by traditional automated microscope stages use lead screws with motors for precise positioning of X-Y coordinates. Z-stack images can be changed by leadscrew and motor or also use features like a liquid lens that can change focal position by applying an electric field to the liquid lens for extremely fine positioning. It is possible to move the sample container, the camera, or both. Traditionally, moving the container and fixing the optics supports stability and protects against vibrational blur if the optics were to move.

### Methods

Methods of the disclosure include detecting the presence or amount of sample components in a biological sample. The methods include processing a native sample to suspend the sample in a liquid suspension medium by mixing the sample with surfactants and pH adjusting agents as described herein. Once the components are suspended into a homogeneous liquid sample, the components can be stained with a fluorescent stain or other stain. For example, bacterial stains can be selected as described herein such that the stain provides a sufficient signal to noise ratio and provides minimum optical background interference to avoid false positive bacteria identification. In some embodiments, all of the non-sample ingredients of the suspension liquid are in solid form and mixed with a diluent (e.g., water or buffer) and the native sample to provide the liquid sample.

After the staining of sample components, the sample may be added to a sample container having sections with different Z-axis heights that allow for settling of components of the liquid sample to settle for a predetermined amount of time. Following a predetermined amount of time, an image of the sample is obtained at a location comprising a unique sets of X-axis and Y-axis coordinates within a FOV of an increment of a Z-axis height (Z-stack image) of one or more sections of the sample container. The presence or absence of sample components within a FOV is determined and may be correlated to the presence or amount of the component in the sample container and/or raw sample. In another embodiment, a plurality of FOVs can be imaged at the same or different set of X-Y coordinates within each section of sample at different times.

The Z-axis depths of the various sections of the sample container determine when all sample components have settled on the base of each section. Commonly found cells (such as red or white blood cells) usually settle at a rate of around 1 μm per second. Therefore, in various embodiments, a predetermined amount of time will allow the components of the sample to settle at least enough such that the FOVs imaged by the apparatus of the disclosure will have minimal or no sample components other than a single type of component, e.g., bacteria, as all or most other components will have settled to the bottom of a section of the container or at least below the range of FOVs imaged according to the disclosure. In shallower sections of the container, components will settle on the bottom of a section in a shorter period of time than in a deeper section.

A set of Z-stack images (capturing successive images vertically through the bulk of a liquid sample in various sections) can be captured in a camera attached to the microscope. In some embodiments, imaging can begin before settling of sample components is complete by capturing a set of Z-stack images (capturing successive images vertically through the bulk of a liquid sample). The images can be analyzed and the data can be compared as histograms to differentiate the sample components (e.g., bacteria from the non-bacteria). For example, intensity values from scans of different Z-stack increments of the liquid sample intensity may be plotted as a histogram with the x-axis being depth in the chamber. The histograms may be used to develop algorithms to classify/quantify bacteria. In addition, a time series may be added to incorporate the settling profiles of sample components.

Under a fluorescence microscope, the stained sample components, (e.g., bacteria) can be seen with an appropriate optical fluorescence filter set based on the different excitation and emission wavelengths associated with the component stains. Image analysis would have minimum optical background interference to avoid false positive component identification since some components in the sample will settle more rapidly than others (e.g., blood cells settle faster than bacterial cells). In one embodiment for the analysis of bacteria, Z-dimension dispersion in a bulk fluid, a full or partial three dimensional Z-stack image rendering can be obtained for further analysis. In addition, optical image capture approaches can be applied to provide high image quality for better bacteria evaluation accuracy. In various aspects, these are high pixel resolution cameras with higher quantum efficiency and high-speed light shutters as well as confocal scanning or spinning-disk setting.

An image viewer program can adjust the contrast of the image to an optimal intensity allowing better visualization of sample components including, for example, fluorescence-stained bacteria. Additional brightfield image acquisition may be used to analyze and screen out non-specific fluorescence staining objects such as cell debris. Each unique bacteria Z-dimension dispersion can be evaluated. The dispersion gradient is based on the container depth, the type of bacteria (rod or cocci), and the component concentrations. The gradient dispersion will be used to count and differentiate the different types of sample components. For example, zooming in on bright-field images may allow for distinguishing bacterial rods from cocci. Cocci tend to be one or a few pixels and will have a roughly circular shape. Rods will have an elongated shape (naturally bigger than cocci) and are identified from cocci based on that elongation.

In some embodiment the FOV collected for a Z-stack image is above the settled elements at the bottom of the sample container. As long as the bottom of the container is sufficiently out of focus (invisible), reliable Z-stack images can be obtained. In another embodiment, the entire bulk sample can be scanned, including the bottom of the container.

In various embodiments, an initial scan can be performed immediately once the container is loaded and then repeat scans can be performed at set time intervals to compare differences that can provide inferences about the bacteria supporting separation from non-bacteria and typing using traditional or machine learning algorithm techniques.

By capturing brightfield and fluorescent images at each location, the method of the disclosure increases the accuracy of the identifying features of sample components (e.g., bacterial shape). The fluorescent signal can be reduced to single parameter, such as magnitude of fluorescence intensity, or it can be used in using machine learning techniques to extract the needed information for classification. For example, machine learning approaches can evaluate images and not just mathematical extractions from images. Therefore, it is not necessary to use classical computer vision approaches to extract attributes from images that provide quantitative values that can then be used for classification. It is possible to use techniques that can include convolutional neural networks that can evaluate images that match reference images. Alternatively, machine learning techniques can use training data sets are used to indicate that bacteria is present in the sample and allow the machine learning logic to find the appropriate attributes as part of training and then provide a metric for the various sample components.

In one example, the number of FOVs imaged is optimized to ensure that a minimum sample count for each component of interest (e.g., cell). Optimization is based on random distribution and sampling statistics. Each component is counted and analyzed in the predetermined number of FOV to report results. In some aspects, the absolute counts per microliter for any component is not determined if the system is not calibrated in a manner to support that type of analysis. Instead, relative statistics are performed to compare specific component counts to other component counts, such as total RBC or total WBC to provide a reference.

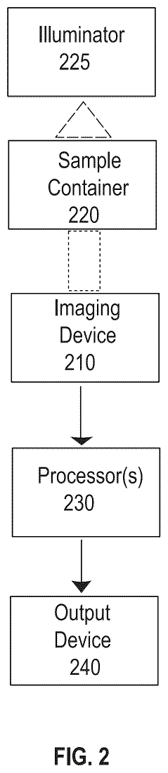

Referring now to FIG. 2, a block diagram of various components is shown. The components include an imaging device 210, a sample container 220, an illuminator 225, one or more processor(s) 230, and an output device 240. In embodiments, the components 210-240 may be co-located in a single apparatus, such as in a point-of-care apparatus of a veterinary clinic. In embodiments, one or more of the components may be located in different systems or devices. For example, one or more of the processor(s) 230 may be a processor in a cloud system. In embodiments, the components may include a communication device (not shown) for communicating information between different systems or devices.

The imaging device 210 is configured to capture a field of view containing at least a portion of the sample container 220. In particular, the sample container 120 includes a sample chamber, and the imaging device s10 captures a field of view containing at least a portion of the sample chamber. The sample chamber is illuminated by the illuminator 225, which may include a brightfield illuminator and/or a light source which induces Stokes shift and causes stained sample components to fluoresce. An example of a light source which induces Stokes shift and causes stained sample components to fluoresce is a blue light LED (light emitting diode) or an ultraviolet light LED. Such light sources may be implemented using a brightfield illuminator together with one or more filters configured to pass only blue light. Known staining methods may be used in order to cause the sample components to fluoresce. In embodiments, the illuminator 225 may be positioned relative to the sample container 220 in different ways and may include separate components that have different positions relative to the sample container.

In embodiments, the sample container 220 is movable to enable the imaging device 110 to capture different fields of view containing at least a portion of the sample chamber. In embodiments, rather than the sample container 220 moving, the imaging device 210 is movable to capture different fields of view containing at least a portion of the sample chamber.

Capturing multiple fields of view will be described in more detail later herein. For now, it is sufficient to note that each field of view captures at least a portion of the sample chamber of the sample container 220. In embodiments, an entire cross-sectional area of a sample chamber of the sample container 220 corresponds to more than one-hundred fields of view, such as in the range of two-hundred to four-hundred fields of view, among other possibilities. In embodiments, the entire cross-sectional area of the sample chamber in the sample container 220 corresponds to less than one-hundred fields of view. In embodiments, the imaging device 210 and/or the sample container 220 move at a rate such that between fifty to one-hundred different fields of view are captured each minute. In embodiments, other rates of capturing fields of view are within the scope of the present disclosure.

With continuing reference to FIG. 2, the processor(s) 230 are configured to analyze the images captured by the imaging device 210. As an example, in embodiments where the illuminator 225 is a brightfield illuminator, any RBCs in the images would appear in color and any WBCs in the images would appear in a different color. As another example, in embodiments where the illuminator 225 is a light source that induces Stokes shift and causes any stained WBCs to fluoresce without causing any RBCs to fluoresce, the images would only show the fluorescing WBCs. In embodiments, the illuminator 225 contains a brightfield illuminator as well as a light source that causes fluorescence. In embodiments, brightfield images and fluorescence images may be combined to provide composite images.

In embodiments, sample components (e.g., various types of cells or bacteria, etc.) may be imaged by brightfield imaging and/or fluorescence imaging, and machine learning models and/or image analytics may be applied to detect one or more constituents in such images. In embodiments, brightfield images and fluorescence images may be combined to provide composite images. In embodiments, other types of images are contemplated to be within the scope of the present disclosure.

The processor(s) 230 count number of a first sample component in the images and, optionally, may count number of a second sample component in the images, among other possible components. In embodiments, the processor(s) 230 may also apply machine learning models and/or image analytics to detect sample components. In embodiments, the processor(s) 230 may also apply machine learning models and/or image analytics to detect various component morphologies (for example, left shift (immature neutrophils), toxic change, reactive lymphocytes, among other morphologies). Known methods can be used to implement such machine learning models and/or image analytics.

The processor(s) 230 causes the output device 240 to provide information regarding components to a person or user, such as number of components counted and/or detected, component morphologies, among other possible information. The output device 240 may be any output device capable of communicating information to a person or user. In embodiments, the output device 240 is a display panel of a point-of-care device and is in the same device as the other components 210-230. In embodiments, the output device 240 may be an office computer or smartphone of a clinician, and a network device (not shown) may communicate the component information to the office computer or smartphone for display. For example, the processor(s) 230 may cause a text message or an email, that contains the component information, to be sent, and the output device 240 may receive and display the text message or email to a user. Other types of output devices 240 are contemplated to be within the scope of the present disclosure, such as audio output devices, among other possibilities.

FIGS. 1 and 2 are merely an example of some components of an apparatus of the disclosure. It should be understood that an apparatus will include other components not shown in either FIG. 1 or FIG. 2, such as a power supply, memory, electronic storage, network device, and/or other components. Such components, and other variations of an apparatus, are contemplated to be within the scope of the present disclosure.

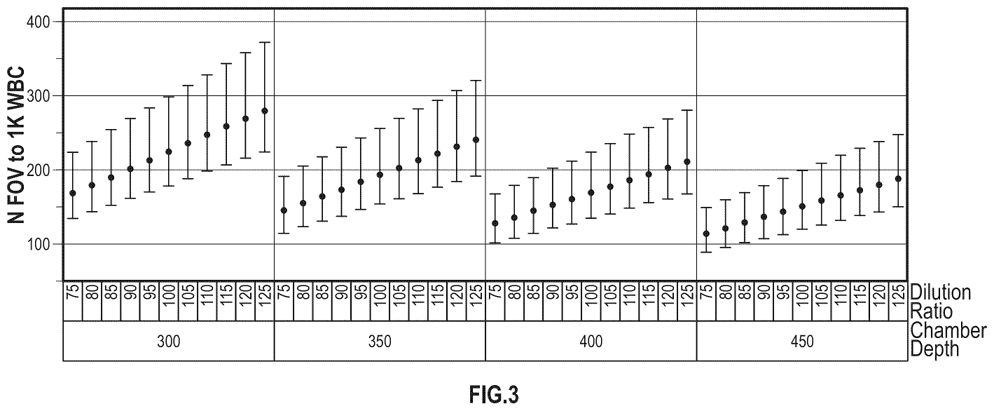

In accordance with aspects of the present disclosure, the number of fields of view that need to be captured by an imaging device to reach a statistically confident count of sample components corresponds to the depth dimension of the sample chamber (or of the sample chamber region) that contains the components. FIG. 3 shows an example of data relating to counting a white blood cells (WBC) as a sample component using the arrangement shown in FIGS. 1 and 2. As shown in FIG. 3, for a particular ratio of volume of diluent to volume of biological sample (referred to as “dilution ratio”), the requisite number of WBCs may be counted using a fewer number of fields of view when the depth dimension is greater. For example, for dilution ratio of 75:1, the number of fields of view needed to count 1,000 WBCs in a 300 micrometer depth region is in the range of about 135-225 fields of view. In comparison, for the same dilution ratio of 75:1, the number of fields of view needed to count 1,000 WBCs in a 450 micrometer depth region is in the range of about 90-150 fields of view.

Also, as shown in FIG. 3, the number of fields of view that need to be captured by an imaging device to reach a statistically confident count of white blood cells (e.g., at least 1,000 WBCs) corresponds to dilution ratio of the mixture that contains the white blood cells. For a particular depth dimension, the requisite number of WBCs may be counted using a fewer number of fields when the dilution ratio is smaller. For example, for a 300 micrometer depth region, the number of fields of view needed to count 1,000 WBCs given a dilution ratio of 125:1 is in the range of about 220-375 fields of view. In comparison, for the same 300 micrometer depth region, the number of fields of view needed to count 1,000 WBCs given a dilution ratio of 75 is in the range of about 135-225 fields of view.

As demonstrated by the example data of FIG. 3, an imaging device does not need to capture the same number of fields of view for chambers or regions with different depth dimensions and/or for different dilution ratios. While the data of FIG. 3 is merely an example, the trends shown by the example of FIG. 3 will persist for different biological samples that settle. While the data of FIG. 3 shows a general trend of fewer fields of view for greater depth dimensions and lower dilution ratio, the data also reflects a range of fields of view for each combination of depth dimension and dilution ratio. The ranges of fields of view for different combinations of depth dimensions and dilution ratios may overlap, such that in various scenarios, number of fields of view may be greater for greater depth dimensions and/or for lower dilution ratios and may be smaller for lower depth dimensions and/or for greater dilution ratios. Such embodiments are within the scope of the present disclosure.

Generally, the number of fields of view depends on the concentration of the constituents to be identified and the number of constituents to be counted. The number of fields of view can be determined based on a depth dimension and a dilution ratio that are optimized for the number of constituents to be identified and counted and for their nominal concentrations. However, if the operation looks for different constituents in a shallower region than in a deeper region, with a higher nominal concentration in the shallower region, it is possible that more or fewer fields of view may be captured in the deeper region than in the shallower region. Such embodiments are within the scope of the present disclosure.

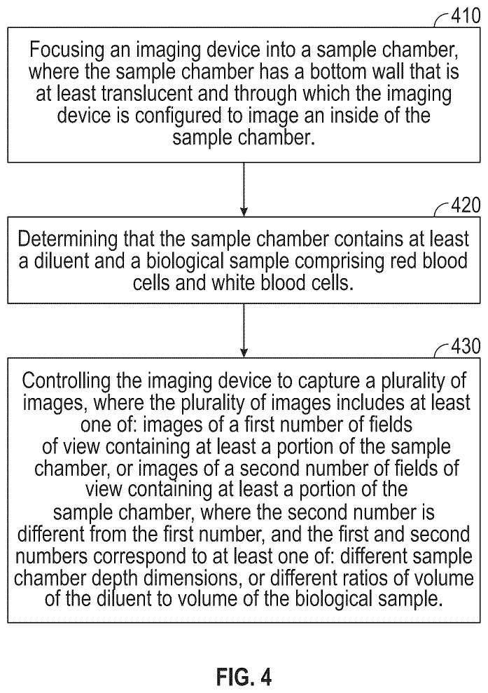

In accordance with aspects of the present disclosure, FIG. 4 is a flow diagram of an operation for evaluating a biological sample containing WBCs and RBCs. At block 410, the operation involves focusing an imaging device into a sample container where the sample container has a bottom wall that is at least translucent and through which the imaging device is configured to image an inside of the sample chamber. In embodiments, the imaging device may be the imaging device 420 of FIG. 4 or may be another imaging device.

At block 420, the operation involves determining that the sample chamber contains at least a diluent and a biological sample comprising red blood cells and white blood cells. The contents of the sample chamber may be determined in various ways. In embodiments, the contents of the sample chamber may be determined by scanning a code on a sample container, such as scanning a barcode or an alphanumeric code, among other possible codes. The scanner for scanning the code may be fixed within a point-of-care apparatus or may be a separate device, such as a camera of a smartphone, which can communicate a result of the scan to a point-of-care apparatus. In embodiments, a user of a point-of-care apparatus may manually indicate the contents of the sample chamber, e.g., by selecting an option on a user interface. Such and other embodiments are contemplated to be within the scope of the present disclosure.

At block 430, the operation involves controlling the imaging device to capture a plurality of images, where the plurality of images includes at least one of: images of a first number of fields of view containing at least a portion of the sample chamber, or images of a second number of fields of view containing at least a portion of the sample chamber, where the second number is different from the first number, and where the first and second numbers correspond to different sample chamber depth dimensions and/or different ratios of volume of the diluent to volume of the biological sample. That is, the imaging device captures different numbers of fields of view depending on the sample chamber depth dimension(s) and/or the dilution ratio, as exemplified by the data of FIG. 3.

As described above, in embodiments, a sample chamber may include two regions that have different depth dimensions. In such embodiments, the operation of block 330 captures images of a first number of fields of view for a first region of the sample chamber having a first depth dimension and captures images of a second number of fields of view for a second region of the sample chamber having a second depth dimension. If the dilution ratio is the same in the two regions, the first and second numbers of fields of view correspond to the different sample chamber depth dimensions and to the dilution ratio. For example, in cases when the second depth dimension is greater than the first depth dimension, the second number of fields of view may be less than the first number of fields of view. If the dilution ratio is different in the two regions, the first and second numbers of fields of view correspond to the different sample chamber depth dimensions and to the different dilution ratios.

As described above, in embodiments, a sample chamber may include more than two regions that have different depth dimensions, such as three regions that have different depth dimensions. In such embodiments, the operation of block 330 captures a first number of fields of view for a first region of the sample chamber having a first depth dimension and captures a second number of fields of view for a second region of the sample chamber having a second depth dimension. Additionally, in the example of three regions, the operation of FIG. 3 captures a third number of fields of view for a third region of the sample chamber having a third depth dimension. If the dilution ratio is the same in the three regions, the first, second, and third numbers of images correspond to the different sample chamber depth dimensions and to the dilution ratio. For example, in cases when the third depth dimension is greater than the second depth dimension, which is greater than the first depth dimension, the third number of fields of view may be less than the second number of fields of view, which may be less than the first number of fields of view. If the dilution ratio is different in the three regions, the first, second, and third number of fields of view correspond to the different sample chamber depth dimensions and to the different dilution ratios. In embodiments, the first number of fields of view may be captured before capturing the second number of fields of view, which may be captured before capturing the third number fields of view. This approach allows constituents in the regions having greater depth dimensions to settle more or to fully settle before they are imaged.

For four or more regions in a sample chamber, the same principles described above apply to such sample chambers.

In aspects of the present disclosure, the captured images may include brightfield images and/or fluorescence images. In embodiments, brightfield images and fluorescence images are captured in a region. For example, multiple fluorescence images are captured in a higher depth dimension region (e.g., 300 micrometers or 400 micrometers) to optically erase the RBC and provide images showing WBC. Then, brightfield images can be captured only in the fields of view where a fluorescent image indicated a WBC is present. In embodiments, one or more regions may be captured using brightfield images, and one or more regions may be captured using fluorescence images. For example, brightfield images may be captured in regions where constituents would be less crowded, such as in smaller depth dimensions (e.g., 100 to 200 micrometers), and fluorescence images may be captured in regions where constituents may be more crowded, such as in larger depth dimensions (e.g., 200 micrometers or greater). An example is described farther below herein.

In accordance with aspects of the present disclosure, the fields of view captured by the operation of FIG. 3 may by any fields of view containing at least a portion of a sample chamber and may be captured using any working distance and depth of field. Each image captures a field of view. Working distance refers to the object to lens distance where the image is at its sharpest focus, and depth of field reflects the distance between the nearest and furthest elements in a captured image that appear to be acceptably in focus. For example, if a sample chamber depth dimension is one-hundred micrometers and the working distance is within the sample chamber, then a depth of field of one-hundred micrometers or larger would result in all depths within the sample chamber being in acceptable focus in the field of view. Depth of field and what is considered “acceptable” focus will be understood in the field of optical imaging. In contrast, if the depth of field is less than one-hundred micro-meters, then some depths within the sample chamber would not be in acceptable focus in the field of view. In the latter case, the lens of the imaging device would need to be moved in the depth direction (e.g., Z-direction) to bring other portions of the depth within the sample chamber into acceptable focus. In the former case, because the entire depth within the sample chamber is in acceptable focus, the imaging device and/or the sample container may be moved in the non-depth directions (e.g., X- and Y-directions) to capture different constituents.

In embodiments, the operation of block 330 in FIG. 3 captures at least the bottom of the sample chamber in acceptable focus. In embodiments, the first number of images and the second number of images captured in block 330 include images focused on a plurality of depths in the sample chamber. For example, for a depth of field of twenty micrometers and a sample chamber depth dimension of one-hundred micrometers, images may be captured by focusing on five depths in the sample chamber, such as focusing on depths of 10, 30, 50, 70, and 90 micrometers. Such images focused on the five depths would capture an entire column of a 100-micrometer sample chamber.

As mentioned above, an imaging device and/or a sample container may move at a rate such that between fifty to one-hundred different fields of view are captured each minute. In embodiments, other rates of capturing fields of view are contemplated. Based on the rate of capturing fields of view and the optical parameters (e.g., working distance, depth of field), it is possible to capture a sequence of field of views to count a requisite number of sample components within a reasonable time period. In the case of a point-of-care apparatus in a veterinary facility, the amount of time deemed reasonable for acquiring a sample, preparing it in a sample container, and analyzing the sample to provide a result, may vary, but a typical veterinarian visit may last no longer than thirty minutes. Therefore, as part of such a visit, a reasonable time period for a point-of-care apparatus to capture images of a sample chamber and analyze them may be less than ten minutes, for example. Counting sample components involves using object detection and/or image analytics, as described above. When a requisite number of sample components has been counted, the imaging operation may be stopped.

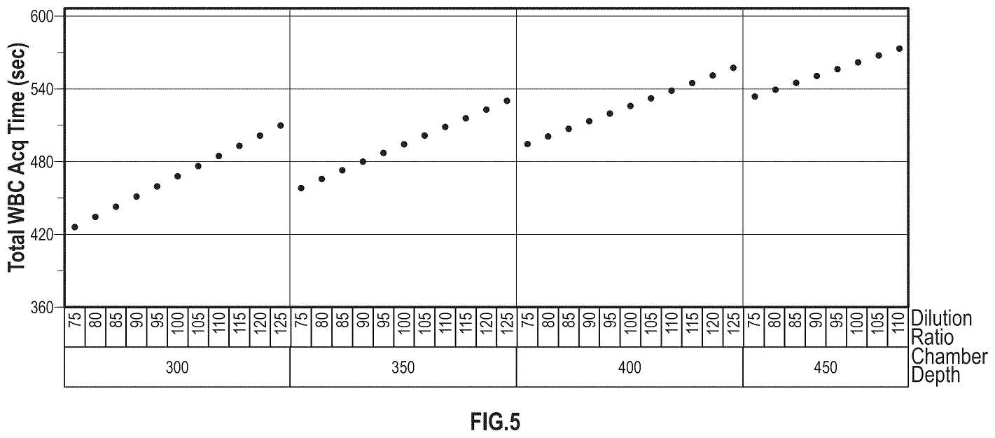

As an example of amount of time needed to conduct the counting, FIG. 5 shows an example of data relating to total time needed for cells to settle to the bottom of a chamber and to count 1,000 WBCs using the arrangement shown in FIG. 4.

For a chamber depth dimension of 300-micrometers, a constituent settling at 1-micrometer per second will need a settling time of 5 minutes to fully settle. For a chamber depth dimension of 450-micrometers, a constituent settling at 1-micrometer per second will need a settling time of 7 minutes 30 seconds to fully settle. Full settling time can be computed for other channel depth dimensions and other settling rates. Together with full selling time, additional time is required to capture the various fields of view to count 1,000 WBCs, and such additional time varies by dilution ratio. Generally, less time and fewer fields of view are needed to count 1,000 WBCs when the dilution ratio is lower, due to higher density of constituents, and more time and more fields of view are needed to count 1,000 WBCs when the dilution ratio is higher, due to lower density of constituents.

The data of FIG. 5 shows an example of allowing a constituent of interest to fully settle and counting 1,000 WBCs. In embodiments, at least some number of WBCs may be counted before they have fully settled. For example, in embodiments, images may be captured while WBCs are in the process of settling but have not fully settled to the bottom of the sample chamber. If the depth of field is sufficient, WBCs across a range of depths will appear acceptably in focus in the images. Such images may include brightfield images and/or fluorescence image, and WBCs may be detected in the images by object detection and/or image analytics in the manner described above herein. When a requisite number of WBCs has been counted (e.g., 1,000 WBCs), the imaging operation may be stopped. By capturing images while a constituent of interest is settling, the total amount of time needed to count a requisite number of WBCs may be reduced, or a greater number of WBCs may be counted within a reasonable amount of time.

Accordingly, aspects have been described for capturing fields of view containing WBCs and RBCs in a single analysis and counting a requisite number of WBCs and/or detecting various WBC morphologies. The descriptions above are merely examples. In embodiments, rather than counting 1,000 WBCs, the operations can define a maximum number of fields of view and/or a maximum number of images to be captured. Such maximum numbers of fields of view and/or images may be set to values that would generally yield at least 1,000 WBCs. However, a biological sample contains fewer WBC than average, then the captured images may yield less than 1,000 WBCs.

### Data Processing

The presence or amount of sample components can be determined by scanning the sample at various locations and at various times, and comparing the scans of each scan to predetermined images of known sample components present at each location and time in any particular section of a sample.

For any particular sample type, a separate algorithm for image analysis may be developed based upon known and/or expected components in a sample. For instance, ear cerumen samples that have been processed into liquid samples of the disclosure can be expected to have different components that liquid samples containing the components for whole or peripherical blood or fine needle aspirates.

Components of each algorithm can include sample dilutions, predetermined settling times and sample locations of captured images at the determined settling times. Each sample type can be expected to provide distinct set of images where, for each dilution, the images of particular sample locations and times can be compared to images of samples having expected or known components at the particular locations and times.

In embodiments of the disclosure, the imaging/scanning component of analysis of a sample may be completed in less than ten minutes. To increase efficiency, the number of images of a sample can be minimized.

In some embodiments, an algorithm may mask off any fluorescence signal from a source that is too large to be a particular sample component (e.g., bacteria) and then quantifies the magnitude of fluorescence from the remaining image area, evaluating the dispersion of bacteria throughout the image (bacteria should be roughly uniformly distributed and clusters would indicate something else). In another embodiment, an algorithm may consider bacteria size/shape within the image (e.g., by convolutional neural network or other) to determine what is and is not a particular component and then adding them up and comparing with a threshold or reporting quantity per FOV.

In some embodiments, an algorithm, program, or software may be used to quantify the components in a sample. In some embodiments, a computing device coupled to or in communication with the camera executes instructions (e.g., instructions stored in a memory of the computing device) in order to identify, measure and/or differentiate components in the sample. Such instructions may be executed by a processor of the computing device in order to automate a portion of the measurement described above in relation to naked-eye techniques. For example, the instructions may be configured to measure the amount of a component in the sample by measuring or determining a number of pixels that correlates to the component (e.g., corresponds to bacteria cells or an area of the image that consists of a bacteria cells) in an image of the sample. Additionally or alternatively, the instructions may cause the computing device to measure an amount of components in the sample by measuring an area of an image of the sample that includes the components. Measuring the area that includes the components may include determining a number of pixels of an image that relate to the component. Differentiating the components from the background of the image may include analyzing a numerical value associated with the pixels. For example, each pixel in an image could include a value corresponding to an amount of collected light, a level of intensity, brightness, coloration, greyscale, or another optical property of the pixel (e.g., a pixel in 8-bit image could be represented by a number between 0 and 255, where 0 corresponds to black and 255 corresponds to white). In a particular example, a threshold value could be set such that pixels with a value higher than the threshold are considered as comprising the components, while those with a value lower than the threshold value are considered background. In such a case, determining a number of pixels that relate to the components may include determining a number of pixels that are above or below some threshold value.

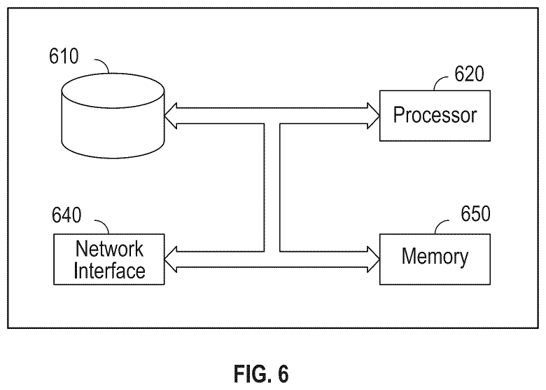

Referring now to FIG. 6, there is shown a block diagram of example components of a point-of-care apparatus at a veterinary facility. The point-of-care apparatus includes an electronic storage 610, a processor 620, a network interface 640, and a memory 650. The various components may be communicatively coupled with each other. The processor 620 may be and may include any type of processor, such as a single-core central processing unit (CPU), a multi-core CPU, a microprocessor, a digital signal processor (DSP), a System-on-Chip (SoC), or any other type of processor. The memory 650 may be a volatile type of memory, e.g., RAM, or a non-volatile type of memory, e.g., NAND flash memory. The memory 650 includes processor-readable instructions that are executable by the processor 620 to cause the apparatus to perform various operations, including those mentioned herein, such as the operations shown and described in connection with FIGS. 2 and 4, and/or applying machine learning models or image analytics, among others.

The electronic storage 610 may be and include any type of electronic storage used for storing data, such as hard disk drive, solid state drive, and/or optical disc, among other types of electronic storage. The electronic storage 610 stores processor-readable instructions for causing the apparatus to perform its operations and stores data associated with such operations, such as storing data relating to computations and storing captured images, among other data. The network interface 640 may implement wireless networking technologies and/or wired networking technologies.

The components shown in FIG. 6 are merely examples, and persons skilled in the art will understand that a coordination system includes other components not illustrated and may include multiples of any of the illustrated components. Such and other embodiments are contemplated to be within the scope of the present disclosure.

### EXAMPLES

The Examples that follow are illustrative of specific embodiments of the disclosure. They are set forth for explanatory purposes only, and are not intended to limit the scope of the disclosure.

### Example 1

A sample containing cocci strain Staphylococcus aureus was analyzed according to the method of the disclosure. The about 107/mL bacteria sample was harvested from an agar plate and suspended in about 1 ml of a suspension liquid containing 7.9 g/L of sodium chloride, 1.65 g/L of Tris-HC1, 0.8 g/L of EDTA and 2.35 g/L of Tris-base. SYTO-13™ dye was used and prepared at the final concentration of 2.5 μM to stain the sample.

Stained sample was loaded on an Incyto™ cell counter slide as a sample container. A Nikon Ti microscope with a confocal scan head was used to capture the Z-stack images in 5 μm fields of view (FOV).

FIG. 6 is a gray-scale reproduction of a Z-stack image having a depth of 100 μm (insert A) and a Z-stack dispersion of 60 μm of Z-stack increments of 5 μm within the 100 μm depth. Each object represents one coccus, which in the original image is shown as a green fluorescent signal. Insert B of FIG. 3 shows a plane of Z-stack images close to the container bottom. Insert C of FIG. 3 shows a plane of Z-stack images close to the cell counter top. Inserts B and C show a concentration difference suggesting a gradient dispersion that is a function of settling time and can provide insight regarding the bacteria based on its time-based vertical concentration histograms.

### Example 2

An experiment to compare the difference in Z-dispersion for both cocci and rod was conducted using the same conditions as those used in Example 1, except the bacteria was Serratia marcescens, which are rods.

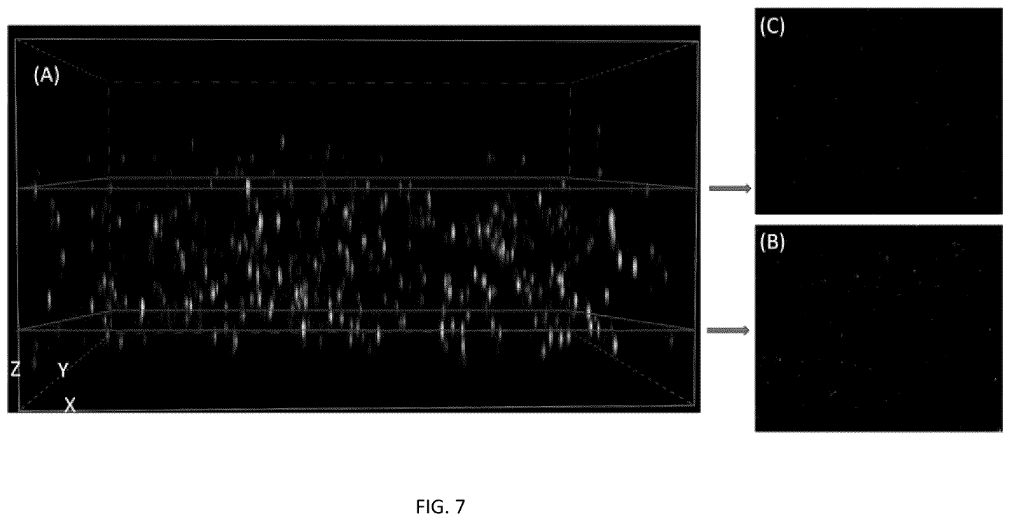

FIG. 7 show a 3-D dispersion in a sample having a Z-stack depth of 100 μm and the Z-dispersion is about 70 μm. Each object in Insert A represents one rod. Insert B shows a plane of Z-stack images close to the cell counter bottom. Insert C shows a plane of Z-stacks image close to the cell counter top. Each fluorescence-stained rod can be seen as an individual population.

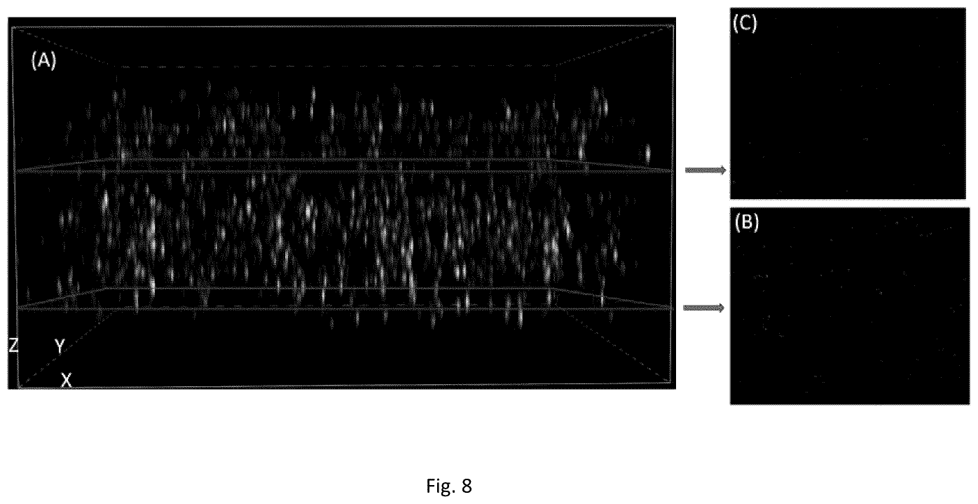

Both cocci (FIG. 7) and rod (FIG. 8) have different Z-dispersion properties and could be used to differentiate the type of bacteria. While not particularly shown in FIG. 7, enlarged brightfield images can show distinguishable images of rods and cocci. Rods will have an elongated shape (naturally bigger than cocci) and are identified from cocci based on that elongation.

### Example 3

To test the unique bacteria Z-dispersion, an E. coli containing liquid sample was prepared to a concentration at about 10⁷/mL bacteria in deionized water and compared to a liquid sample containing dust with similar size and concentration. Bacteria and dust samples were filled in separate Incyto™ cell counter slides as sample containers. Sample imaging was performed on a Nikon Ci microscope with a brightfield image acquisition process. The Z-scanning range with brightfield imaging was set to include the top and bottom of a container. Z-step images of 5 μm each were collected and rendered into a 3-D volume view.

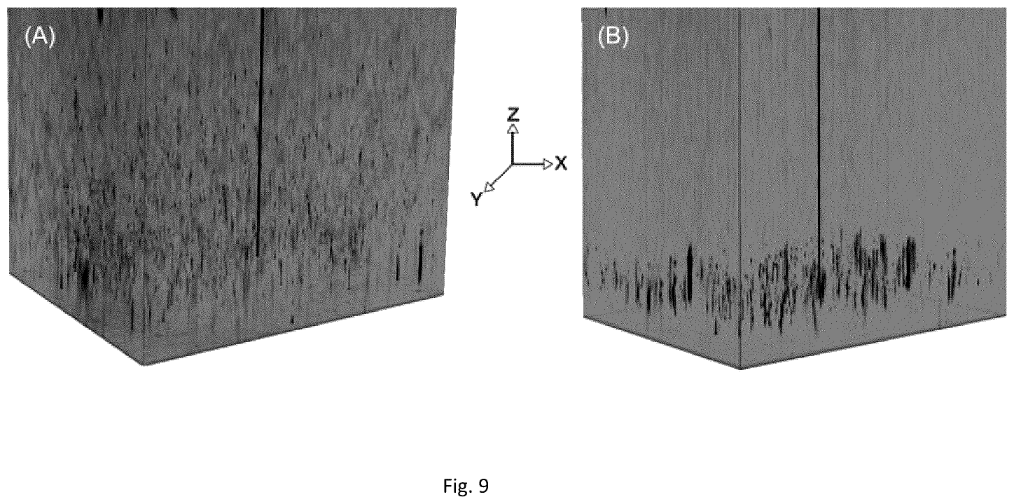

FIG. 9 shows a composite image of the entire sample container sample with either (A) E. coli, or (B) dust, after a predetermined amount of time of twenty minutes. Each black colored object represents a bacteria or dust population. The dust settled close to the cell counter bottom after the predetermined amount of time.

### Example 4

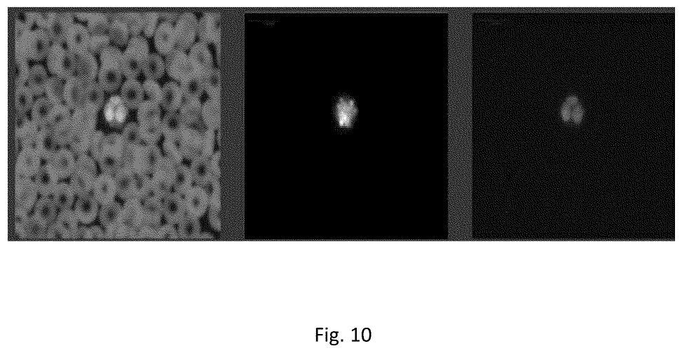

FIG. 10 shows bright field and fluorescent images of the bottom of a sample container where components of a blood sample has settled. In the bright field image (left panel), a white blood cell is shown in a sample also containing red blood cells, typically in a ratio of one white cell to each 1000 red cells. Because white blood cells fluoresce and red cells do not, the white cell can be identified using a fluorescent light source (middle panel). The left image is a composite image including fluorescent and brightfield images and therefore has visible red blood cells The fluorescent images (middle and right panels) show that the red blood cells become invisible in their view and support white cell interrogation. The center image is the blue-excited image while the right image is the UV-excited image. The nucleus features are shown more clearly in the right image based on the DNA stain applied, while the center image lights up both RNA and DNA and provides a better picture of the rest of the cytoplasm beyond the nucleus.

The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A method for identifying a component in a liquid sample, the method comprising:
diluting a native sample or non-dissolving components thereof with a dilution liquid to provide a liquid sample having a uniform component distribution;
introducing the liquid sample into a sample container comprising a first section comprising a first Z-axis height and second section comprising a second Z-axis height that allow for settling of components in the liquid sample;
allowing the components in the liquid sample to settle for a predetermined amount of time; and
imaging a Z-axis field of view (FOV) comprising an increment of the Z-axis height of the liquid sample at a first location within the first section and a second location within the second section, wherein the first location comprises a first set of X-axis and Y-axis coordinates and the second location comprise second set of X-axis and Y-axis coordinates.

2. The method of claim 1, wherein the native sample is a highly viscous or semi-solid sample biological, environmental or industrial sample.

3. The method of claim 1, wherein the diluting comprises mixing the native sample with a reagent comprising a surfactant and a buffer.

4. The method of claim 1, wherein the native sample comprises a fine needle aspirate of a tissue.

5. The method of claim 4, wherein the tissue comprises cells from a lymph node, a cutaneous or subcutaneous mass or an organ.

6. The method of claim 1, wherein the native sample comprises ear cerumen.

7. The method of claim 1, wherein the native sample comprises a fecal sample, a lung lavage, a fluid from an effusion, a blood sample, synovial fluid, thoracic fluid, abdominal fluid, cerebrospinal fluid or a urine sample.

8. The method of claim 1, wherein the settling of non-bacterial components in the liquid sample is complete after the predetermined amount of time.

9. The method of claim 1, wherein the settling of non-bacterial components in the liquid sample is incomplete after the predetermined amount of time.

10. The method of claim 1, wherein the Z-axis heights of the first section and the second section are independently between about 0.1 to 0.5 mm.

11. The method of claim 1, wherein a depth of field of the increment of the Z-axis height is about 1 μm to 10 μm.

12. The method of claim 1, wherein a Z-axis height of the first section is at least 50 μm more than the Z-axis height of the second section.

13. The method of claim 1, the imaging is conducted with a confocal microscope or and epifluorescence (EPI) microscope.

14. The method of claim 1, wherein the increment of the Z-axis height comprises a bottom of the first section or the second section of the container.

15. The method of claim 1, wherein the imaging comprises imaging FOVs at a plurality of Z-axis increments in each section.

16. The method of claim 1, wherein the imaging comprises imaging FOVs at a plurality of X-Y coordinates in each section.

17. The method of any of claim 15, wherein the number of Z-axis FOVs is determined by at least one of the component being analyzed, the dilution ratio and a Z-axis height.

18. The method of claim 17, wherein the number of Z-axis FOVs is further determined by a predetermined image acquisition time.

19. The method of any of claim 15, wherein the statistically analyzing comprises correlating a type, presence or amount of a component in the plurality of FOVs to the type, presence or amount of the component in the liquid sample.

20. The method of claim 1, wherein the statistically analyzing comprises correlating a type, presence or amount of a component within a FOV comprises comparing an image from the FOV to a predetermined image indicative of the type, presence or amount of the component.

21. The method of claim 1, wherein the imaging comprises determining a fluorescent response of a stained component in the FOV.

22. The method of claim 1, further comprising obtaining a bright-field image of the FOV.
